(19) United States
(12) Patent Application Publication
(10) Pub. No.: US 2008/0178495 A1
(43) Pub. Date: Jul. 31, 2008

(54) Foot Clip

(76) Inventors: Edward S. Stein, Clayton, MO (US); Terrance J. Mueller, Wright City, MO (US)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 11/668,203

(22) Filed: Jan. 29, 2007

(51) Int. Cl. A61F 5/14

(52) U.S. Cl. 36/140

(57) ABSTRACT

An orthotic device designed to attach directly to the foot of a user as opposed to being placed directly in a shoe. The device is designed to clip to the base of the foot and may be used in conjunction with any type of footwear lacking correct foot support, or may be used without any other footwear at all. The clip will generally include a main body which is formed to extend around the sides of the user's foot and across the base and is frictionally held in position to provide support as the user walks or otherwise uses his or her feet and/or to provide control of hypermobile (abnormally pronating) feet.

### BACKGROUND

1. Field of the Invention

The invention relates to an orthotic device, more specifically to an orthotic device which attaches directly to the foot.

2. Description of the Related Art

It has been estimated that virtually every American, at some point in his or her lifetime, will have a major foot ailment. While many of those ailments will be the result of traumatic injury from work or recreational activities, many more will be caused by a simple ignorance of basic foot care.

Many people regularly wear both poorly fitting and improperly designed shoes. Modern shoes are, more often than not, designed to meet fashion standards that are unrealistic, if not downright unsuitable, for most people's feet. They are not designed to provide the foot with the comfort, protection, and support desirable for proper foot care. For example, many modern shoes for women have heels that are much too tall to be good for the feet, provide for a foot area that is too cramped for the feet to operate properly, and lack even basic support structures to prevent damage to the feet from walking or other activity.

The human foot is a very complex mechanism having many joints, ligaments, and muscles. Every step taken by a human being requires a complex interaction between the foot and the surface on which the human is stepping to both provide for a solid gait and allow the walker to maintain balance. The average human foot will, in just one day, move the equivalent of 187 tons of weight simply by virtue of a person's movement. If footwear is not designed correctly, that weight can be improperly distributed leading to damage and pain.

Properly designed footwear serves to both support the correct movement of the foot and protect the foot from injury. In particular, well-designed footwear provides and encourages desired motion of the foot, while inhibiting undesirable motion to protect the foot and ankle. Further, well-designed footwear provides support for the structure of the foot, allowing the foot to resist repetitive stress and damage.

Some of the most common foot injuries are caused from wearing improper footwear on a regular basis. Further, failure to wear any footwear can also lead to similar problems. Once damaged, an abnormal foot will require more specialized shoe gear for protection. Maladies such as abnormally flexible feet, heel pain, and repetitive microtrauma to the ligaments and other sensitive tissues of the lower extremities can require even additional protection. Similarly, a lack of proper support can result in excessive pronation, where the foot rolls inward in an incorrect manner resulting in increased stretching of ligaments, or other ailments (such as, but not limited to, abnormal stress of the tendons of locomotion), most of which can lead to chronic pain. While all of the above conditions can all be extremely painful on the feet, foot ailments can also lead to additional problems such as ankle, leg, knee, hip, back or neck pain due to the need to compensate for the improper foot motion, increased stress or torque, or muscle pain in the legs or back from compensation.

Even with all these potential problems, many forms of popular shoes, particularly many summer shoes such as sandals and the now ubiquitous “flip-flops,” lack even the most basic arch and foot support, control, or protection. Previously, those who needed support in poorly designed footwear could use specially designed orthotic devices which went into the shoes and provided specially designed support for the user. These shoe inserts, while effective, rely on the structure of the footwear to stay correctly positioned relative to the foot when in use. However, many sandals, more open shoes, and particularly flip-flops simply lack the basic structures necessary to hold such inserts in place. The problem can be made worse as the devices are often large and unsightly, and dissuade the user from wearing them with particular fashionable shoes in which the devices are visible.

Another alternative is custom fitted and designed shoes. These can provide for additional support and comfort without the need for insoles, and can sometimes be used for more fashionable shoes. However, they can become expensive since each pair of shoes has to be custom designed and constructed. Further, they provide no benefit if a user is barefoot.

### SUMMARY

The following is a summary of the invention in order to provide a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems in the art, described herein are orthotic devices, in particular orthotic devices which attach directly to the foot, which serve to provide for support for the foot and can be used with any type of shoe, or while walking barefoot or nearly barefoot.

Described herein, among other things are orthotic devices comprising: a concave main body, said main body including: a base having a generally polygonal form; a lateral flange located toward a first side of said base; and a medial flange located toward a second side, opposing said first side, of said base; wherein said main body has an inner surface between said lateral flange and said medial flange and said lateral flange and said medial flange both extend upward from said inner surface; and a triplanar post sized and shaped to support to an arch of a foot of a human being, arranged on said inner surface of said base toward said medial flange.

In an embodiment of such an orthotic device the base is subdivided into a medial base and a lateral base, the triplanar post is located on said medial base, and the inner surface of said medial base is arranged at an obtuse angle relative to said inner surface of said lateral base.

In another embodiment of such an orthotic device, the base is generally trapezoidal, the lateral flange is located at a top of said trapezoid, the top of said trapezoid is curved and the bottom of said trapezoid is curved.

In another embodiment there is included a longitudinal rib arranged on each of said lateral flange and said medial flange, the lateral flange has a cross section of a generally “C” shape, the medial flange has a cross section of a generally “C” shape, the triplanar post is removably attached to said inner surface, the main body further includes a metatarsal flange, or the main body further includes a heel flange.

There is also described herein methods for providing support to a human foot comprising providing an orthotic device including an arch support pad; attaching said device directly to said human foot in a temporary fashion; and having a human walk while wearing said orthotic device.

In an embodiment of a method the step of having is performed barefoot or wearing flip-flops.

### BRIEF DESCRIPTION OF THE DRAWINGS

FIG. 1 shows an overhead perspective view of an embodiment of a foot clip for the fight foot.

FIG. 2 shows a side perspective view of the embodiment of FIG, 1.

FIG. 3 shows a cut-through view of the device of FIG. 1 along the line 3-3.

FIG. 4 shows a top view of the device of FIG. 1 indicating a removable and interchangeable arch support control.

FIG. 5 shows a side view of the device of FIG. 1 in place on a human foot.

### DESCRIPTION OF THE PREFERRED EMBODIMENT(S)

The following detailed description illustrates by way of example and not by way of limitation. Described herein, among other things, are embodiments of orthotic devices which are designed to be worn directly on a human foot, either in conjunction with footwear or in the absence of footwear. These devices are generally referred to herein as “foot clips” as they are clipped or attached to the foot directly instead of being an insert for a shoe. The devices are not intended to be permanently attached to the foot, but are designed to attach in a temporary manner. It should be recognized that a foot clip can be used with virtually any type of footwear, including footwear in which other orthotic devices may be used and a number of other types of footwear, such as flip-flops, which are generally unusable with other orthotic devices,

FIGS, 1-4 provide various drawings of an embodiment of a foot clip (100) for the right foot (300) of a human. It would be recognized by one of ordinary skill in the art that foot clips (100) would generally be provided as a pair, one for each foot (300), and not singly as shown here. However, only the foot clip (100) for the right foot (300) is depicted here as the corresponding foot clip (100) for the left foot would generally simply be a mirror image of the one for the right foot (300). It should be recognized that for any individual human user, the pair of foot clips (100) need not be exact mirror images in the same fashion that the user's feet need not be identical mirror images of each other. However, the principle should be apparent.

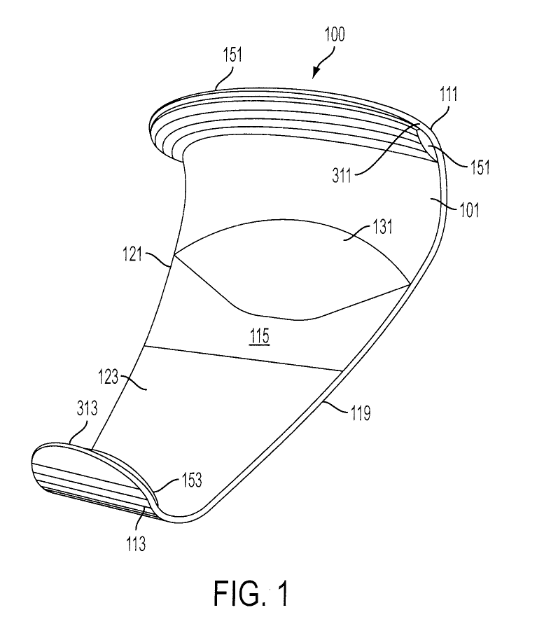

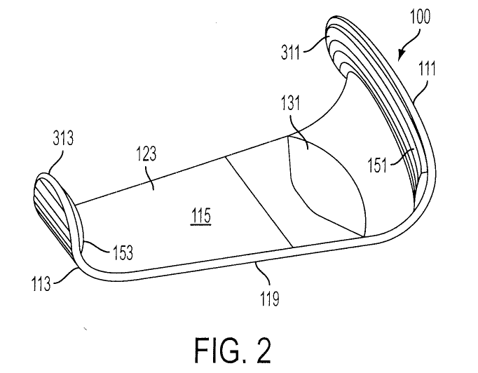

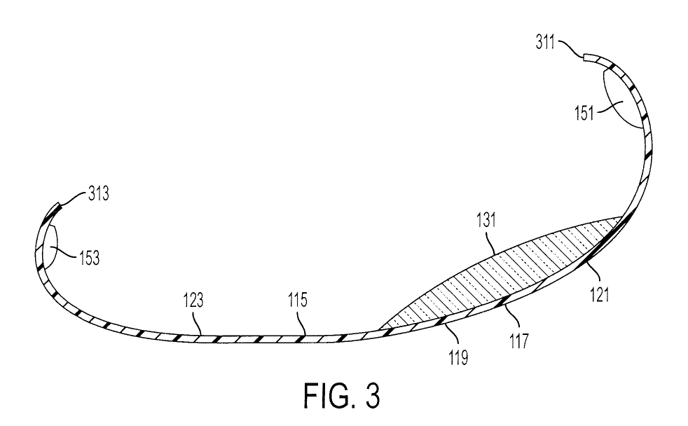

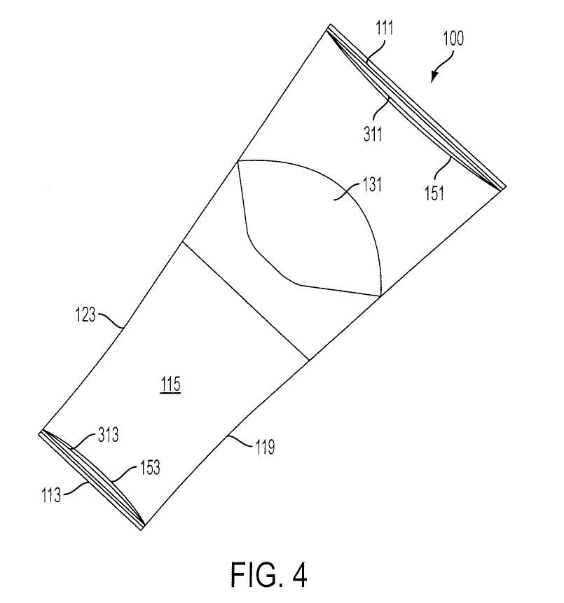

The foot clip (100) of FIGS. 1-4 is a generally one-piece structure formed around a principle component, which provides its shape. The principle component is termed the main body (101). The main body (101) is formed from a generally planar irregular trapezoidal or triangular shape. The main body (101) is bent through various locations in the main body (101) to create the flanges which extend upward from the main body (101). In the depicted embodiment, the two flanges (111) and (113) are formed at the base and top of the trapezoid and provide for medial (311) and lateral ends (313). The resulting structure therefore has a generally concave shape with an inner surface (115) between the two flanges (111) and (113). The structure is such that the foot clip (100) begins at a medial end (311) and ends at a lateral end (313) forming a curving and generally “U” shaped structure between. The wider medial flange (111) tapers to a narrower lateral flange (113). The main body (101) may be comprised of any relatively strong rigid material, including plastic, metal, or related materials, so that it can maintain its shape.

In an alternative embodiment, the main body (101) may additionally include other flanges to provide support for the foot. For instance, in an embodiment, there may be included a metatarsal flange extending underneath the metatarsal region of the foot to support the metatarsal region and/or relieve pressure or inflammation of the metatarsal heads. In a still further embodiment, a heel flange may alternatively or additionally be included to support the heel and/or accommodate heel pain, heel spurs, or planter fasciitis.

Generally, the main body (101) will be sufficiently flexible so as to flex when placed under strain and not injure the foot (300) of the wearer if impacted, but of sufficient rigidity to maintain its shape and provide support for the foot (300) when worn under walking or some running conditions. The main body (101 ) may be of any appearance and in an embodiment will be transparent so as to generally not be visible when worn. In an alternative embodiment, however, the foot clip (100) may be formed to include specific colors or patterns and may, in itself, be used as a fashion accessory. For instance, in an embodiment, the foot clip (100) may be in a color or colors designed to complement a particular color of shoe. In an alternative embodiment, the main body (101) may include patterns which are printed thereon or may include textures or patterns as part of its structure. Any of these embodiments serve to provide a decorative look to the foot clip (100) which can allow it to serve not only its principal purpose of providing support, but also to serve as a fashion accessory.

The main body (101) will generally be relatively smooth on the inner surface (115) so as to be comfortable when worn against the foot (300) for an extended period of time. However, in an alternative embodiment, the inner surface (115) may be textured to provide for a massaging effect or other comfortable surface against the underside of the foot (300) or to help grip the foot (300). The outer surface (117) may also be smooth in an embodiment, or may include texturing or additional components to provide for grip between the foot clip (100) and the sole of a shoe or against the ground.

As is best shown in FIG, 3, the foot clip (100), in an embodiment, will generally have a concave cross section when such cross section is taken perpendicular to the planes of the flanges (111) and (113). In particular, the lateral flange (113) will generally curve around over the inner surface (115) on the lateral end (313). The base (119) of the main body (101) may also include a bend, generally at around its midpoint, dividing the base into two portions comprising a medial base (121) and lateral base (123), in an embodiment. The medial base (121) may then, in turn, be connected to the medial flange (111) which again curves inward over the inner surface (115) as it approaches the lateral end (311). This gives the cross section a generally “U” shape having an obtuse angle between the inner surfaces (115) of the lateral base (123) and medial base (121). The lateral and medial flanges (113) and (111) also have a generally semi circular or “C” or mirrored “C” shape when connecting to their respective base portions.

On the inside surface (115) of the medial base (121) there will generally be attached a triplanar post (131). This triplanar post (131) may be permanently attached to the medial base (121), to the medial flange (111), and/or be removable, depending on the embodiment. It may be of any shape and may be modified to accommodate any bony prominence(s) present on the foot. In an embodiment, the triplanar post (131) is not initially attached to the main body but may be selected for a size and shape appropriate to the user's foot, and may then be adhered to a selected portion of the medial base (121) in a relatively permanent fashion. The triplanar post (131) will generally comprise a wedge of material which is smoothly tapered in a plurality of directions so as to generally conform to the shape of the arch of a human foot. This can result in a wedge of virtually any shape, but in many embodiments the wedge will be triplanar and tapered anteriorly, posteriorly, and laterally. The triplanar post (131) will generally be comprised of a shock-resistant compressible material such as, but not limited to, rubber or foam.

Depending on the embodiment, the triplanar post (131) may be customized to fit a specific foot or formed generally for use with an “average” foot. If custom- formed, the triplanar post (131) may be formed by any method known to one of ordinary skill including plaster casting of the foot as is common in prescription foot orthoses. The triplanar post (131), once designed, may also be used and incorporated into specific foot gear including, but not limited to, shoes. boots, skates; ballet slippers; an ankle/foot orthosis, brace, or other device; or other footwear.

In the depicted embodiment, there is provided at the ends of each of the medial flange (111) and lateral flange (113) a longitudinal rib (151) and (153) which is formed of a “sticky” material, Such materials are generally characterized by having a relatively high coefficient of friction. This material will generally not be permanently adhesive, but instead will be designed to resist an object's shear motion relative to the longitudinal rib (151) or (153). In an embodiment, the longitudinal ribs (153) and (151) may be comprised of tubes, cylinders, or similar shapes. The longitudinal ribs (153) and (151) may be comprised of any material including, but not limited to, plastic or silicone. The longitudinal ribs may be attached or held at the terminal ends (313) and (311) of the lateral flange (113) and medial flange (111) respectively and may be held in place to the foot clip (100) by any method known to one of ordinary skill. In a still further embodiment, the longitudinal ribs (313) and (311) may comprise a strip of adhesive designed to temporarily adhere to human skin which is replaced between uses. The longitudinal ribs (151) and (153) are unnecessary in some embodiments as the general concave design of the main body (101) can provide sufficient tension to keep the foot clip (100) attached to the foot (300).

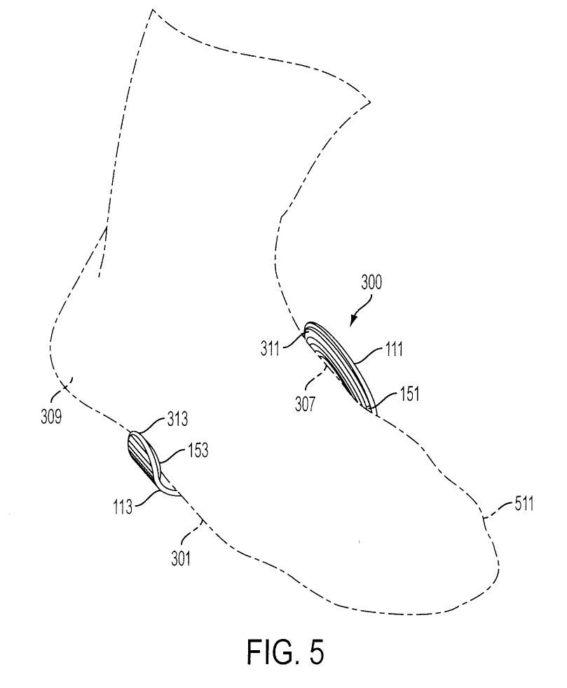

Generally, the foot clip (100) will be worn as shown in FIG. 5. To wear the device, the user will place the main body (101) generally against the underside (301) of their foot (300) with the medial flange (111) arranged toward the inside of the foot (300), the lateral flange (113) arranged toward the outside of the foot (300), and the inner structure (115) adjacent to the sole (301) of the foot (300). The user will then push the foot clip (100) toward his or her foot (300). The structure of the user's foot (300) will generally cause the lateral flange (113) and medial flange (111) to spread apart from each other to allow the foot (300) to pass between the terminal ends (311) and (313) and approach the inner surface (115). As the sole (301) of the foot (300) contacts the inner surface (115), the lateral (113) and medial flanges (111) will follow the foot's (300) natural curvature up the sides (303) of the foot (300) and frictionally engage the sides (303) or top (305) of the foot (300). The foot clip (100) is thus “clipped” to the sole (301) of the foot (300) and will generally be unable to come loose without application of force pulling it away. The “U” shape with the inward curve in the lateral flange (113) and medial flange (111) and the stickiness of the longitudinal ribs (151) and (153) will generally keep the foot clip (101) from moving downward, while the shape of the foot (300) will generally inhibit forward or backward motion. The longitudinal ribs (151) and (153) will also generally resist such forward or backward motion. The foot clip (100) will generally be positioned on the foot so that the medial flange (111) is centered at the medial aspect of the talo-navicular joint area with the triplanar post (131) arranged dorsally and conformed to the inner surface of the foot arch (307) as shown in FIG. 5.

In use, the foot clip (100) may be worn while participating in any activity using the feet for support. Generally, it will be used for normal activity, including walking and running, of the type encountered in daily life. As the foot clip (100) is attached to the foot (300), it can be worn in conjunction with any form of footwear which lacks proper support for the foot (300) as the foot clip (100) can be placed on the foot (300), and then the footwear can be placed on the foot (300). It should also be apparent that the foot clip (100) can be worn when barefoot or otherwise without footwear to provide for support even in this situation. Since the foot clip (100), as previously discussed, can be constructed to be difficult to see when in place or constructed to be fashionable, the clip can also be used in situations where a traditional insole would be unsightly.

Generally, in use, the foot clip (100) will act as a spring and support for the foot (300). In a normal human walking motion, the heel (309) of the foot (300) will generally contact the ground first. The weight of the person is then transferred to the foot (300) which rolls, along the outside of the foot (300), toward the big toe (311). As the person approaches upright, the foot (300) compresses to support his weight. As the step continues, the arch (307) then raises and the foot (300) smoothly rolls upward and outward to support the person's body and continue the forward motion with a final push-off provided by the big toe. Proper amounts of supination in the foot are required for this process to occur properly.

Foot pain is often caused as the foot (300) compresses because, if the arch (307) lacks support, the arch (307) can become unstable (abnormally pronate) and descend toward the ground under the person's weight and forces of acceleration and propulsion. This will result in stretching of the tissue on the underside (301) of the foot (300), forming the arch (307) and related structure, which can result in pain. The triplanar post (131) provides for arch support resisting the arch's (307) motion downward in a step. The foot clip's (100) shape also serves to provide support for the arch (307) through the foot clip's (100) own rigidity and spring. Further, as the lateral base (133) is under the side of the foot (300) touching the ground and compressed into the ground by the foot, pressure from the arch (307) moving downward against the foot clip (100) tilts the clip (100) against the base (301), but such tilt is resisted by the weight of the user. This provides for the foot clip (100) to bend and flex against its concave shape as weight is pushed against it to provide further spring As the step continues, the arch (307) will move upward and the natural resiliency of the main body (101) will generally force the main body (101) to try to reform, keeping the clip (100) attached to the foot (300).

Further, the foot clip (100) can inhibit the foot (300) from other abnormal movement by providing other support to the foot (300). In particular, the foot clip (100) can be used to control or minimize medial column collapse, internal talar and tibial rotation, abnormal pronation, first ray hypermobility, or other common foot problems caused by insufficient support. In effect, allowing the user to have foot support in situations when it would generally not be available is a principle benefit of the foot clip.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art.

1. An orthotic device comprising:
a concave main body, said main body including:
a base having a generally polygonal form;
a lateral flange located toward a first side of said base; and
a medial flange located toward a second side, opposing said first side, of said base;
wherein said main body has an inner surface between said lateral flange and said medial flange and said lateral flange and said medial flange both extend upward from said inner surface, and
a triplanar post sized and shaped to support to an arch of a foot of a human being, arranged on said inner surface of said base toward said medial flange.

2. The device of claim 1 wherein said base is subdivided into a medial base and a lateral base.

3. The device of claim 2 wherein said triplanar post is located on said medial base.

4. The device of claim 2 wherein said inner surface of said medial base is arranged at an obtuse angle relative to said inner surface of said lateral base.

5. The device of claim 1 wherein said base is generally trapezoidal.

6. The device of claim 5 wherein said lateral flange is located at a top of said trapezoid.

7. The device of claim 6 wherein said top of said trapezoid is curved.

8. The device of claim 6 wherein said bottom of said trapezoid is curved.

9. The device of claim 1 further including, a longitudinal rib arranged on each of said lateral flange and said medial flange.

10. The device of claim 1 wherein said lateral flange has a cross section of a generally “C” shape.

11. The device of claim 1 wherein said medial flange has a cross section of a generally “C” shape.

12. The device of claim 1 which said triplanar post is removably attached to said inner surface

13. The device of claim 1 wherein said main body further includes a metatarsal flange.

14. The device of claim 1 wherein said main body further includes a heel flange.

15. A method for providing support to a human foot comprising:
providing an orthotic device including an arch support pad;
attaching said device directly to said human foot in a temporary fashion; and
having a human walk while wearing said orthotic device.

16. The method of claim 15 wherein said step of having is performed barefoot.

17. The method of claim 15 wherein said step of having is performed wearing flip-flops.
